(19) United States
(12) Patent Application Publication
(10) Pub. No.: US 2026/0290605 A1
(43) Pub. Date: Sep. 24, 2026

(54) Methods and Systems for Training and Preparing Machine Learning Model(s) for Use in Identifying a Specific Patient's Future Risk of Developing a Psychiatric Condition

(71) Applicant: Children's Hospital Medical Center, Cincinnati, OH (US)

(72) Inventors: John PESTIAN, Cincinnati, OH (US); Tracy GLAUSER, Cincinnati, OH (US); Daniel SANTEL, Cincinnati, OH (US)

(73) Assignee: Children's Hospital Medical Center, Cincinnati, OH (US)

(21) Appl. No.: 19/476,010

(22) PCT Filed: Apr. 18, 2024
(86) PCT No.: PCT/US2024/025223

§ 371 Date: Oct. 16, 2025

Related U.S. Application Data
(60) Provisional application No. 63/460,206, filed on Apr. 18, 2023.
(60) Provisional application No. 63/584,299, filed on Sep. 21, 2023.
(60) Provisional application No. 63/600,953, filed on Nov. 20, 2023.

(51) Int. Cl. G16H 50/30; G06N 20/00; G16H 10/60

(52) U.S. Cl. CPC G16H 50/30; G06N 20/00; G16H 10/60

(57) ABSTRACT

Early identification of psychiatric conditions such as anxiety and depression in children, adolescents, and young adults coupled with evidenced based treatment can significantly reduce acute and chronic morbidity and complications that occur later in life. An individual with a high risk of developing these serious psychiatric conditions can be identified with data and machine-learning methods before the conditions develop. A method for training and preparing a machine learning model for use in identifying a specific patient's future risk of developing a psychiatric condition (e.g. diagnosis, emergency room visit, hospitalization, suicide attempt) includes the following steps: collecting first electronic health records from previous patients who have been diagnosed with the psychiatric condition; processing the collected electronic health records into a training dataset and training a machine learning model with the training dataset to compute the specific patient's future risks for developing the psychiatric condition (e.g. diagnosis, emergency room visit, hospitalization, suicide attempt).

### CROSS-REFERENCE TO RELATED APPLICATIONS

This application claims the benefit of U.S. Provisional Application Ser. No. 63/460,206 filed Apr. 18, 2023, U.S. Provisional Application Ser. No. 63/584,299, filed Sep. 21, 2023, and U.S. Provisional Application 63/600,953 filed Nov. 20, 2023; the disclosures of which are incorporated herein by reference.

### STATEMENT REGARDING FEDERALLY SPONSORED RESEARCH

This invention was made with government support under Prime Contract No. DE-AC05-00OR22725 awarded by the United States Department of Energy. The government has certain rights in the invention.

### BACKGROUND

The United States is experiencing a mental health crisis. Childhood anxiety (7.8%) and depressive disorders (3.2%) are increasingly common in the U.S. population and have been associated with significant acute and chronic morbidity. Furthermore, these conditions increase the risk of suicide-now the second leading cause of death in individuals aged 10-24 years. Early identification of anxiety and depression in children and adolescents coupled with evidenced based treatment can significantly reduce these pathologies and the complications that occur later in life. However, access to expert and well-trained mental health clinicians who can identify clinical anxiety, depression, and other psychiatric conditions remains limited. As such, empowering primary pediatric clinicians and primary care clinicians with tools trained to identify early children, adolescents, and young adults at high risk for psychiatric conditions could reduce the burden of mental illness for children now and in their future while having profound public health benefits. Early identification poses difficulties. For example, electronic health records (EHRs) may include clinical data collected over time. Those records are highly-dimensional, complex, and heterogeneous, making manual analysis error-prone and posing significant challenges to clinicians in finding relevant information. Accordingly, there is a need for improved technology for identifying and treating anxiety disorders, particularly in the population groups of children, adolescents, and young adults.

### SUMMARY

The technology disclosed herein may be applied in the identification of a specific patient's future risk of developing a psychiatric condition (e.g. diagnosis, emergency room visit, hospitalization, suicide attempt). This future risk can be classified as “high risk,” “low risk,” or “intermediate risk”. The disclosure describes technology that will determine the level of future risk. FIGS. 8A-8C provide three graphs illustrating a visual representation of the disclosure. FIG. 8A is the general output template where the X axis is months in the future using today as the starting point. The Y-axis is the probability that this specific patient will develop a specific psychiatric condition (in this example a diagnosis of an anxiety disorder). The three tiers of cross-hatched horizontal bars represent domain experts' specific interpretation of the risk level for that psychiatric condition. In this example, the bottom hatching region represents a low risk of developing the condition (anxiety disorder), while the top hatching region represents a high risk of developing the condition (anxiety disorder). The middle hatching region represents an intermediate risk of developing the condition (anxiety disorder). FIG. 8B shows a specific patient with a persistent low risk of developing an anxiety disorder. FIG. 8C shows a clear upward trajectory of the probability line indicating an increasing risk for the next 9 months ending with a high risk of developing an anxiety disorder. An embodiment of the technology is currently 80% accurate in identifying patients at high risk for developing an anxiety disorder 6 months before they are diagnosed. An embodiment of the technology is currently 70% accurate in identifying patients at high risk for an emergency room visit or hospitalization for a mental health crisis 30 days before the actual event.

For example, aspects of the disclosed technology may be used to implement a method using an individual's electronic health record. Those methods, combined with the electronic medical records' structured and unstructured data, can be used to determine a plurality of predictive point-in-time and longitudinal data that provide prediction input data for training machine learning models that are used for identifying a specific patient's future risk for developing a psychiatric condition (e.g. diagnosis, emergency room visit, hospitalization, suicide attempt).

Other applications of the disclosed technology demonstrated in FIGS. 8A-8C, including in the form of systems or computer-readable media, correspond to methods such as those described above for other psychiatric conditions such as depression, suicide, and violent outbursts. Additional benefits and advantages of the disclosed technology will also be apparent in view of the following description and accompanying figures. Accordingly, the described technology should be understood as being illustrative only and should not be treated as limiting.

In an aspect, a method for training and preparing a machine learning model for use in identifying a specific patient's future risk of developing a psychiatric condition in human subjects is provided. The method includes the following steps: collecting first electronic health records from previous patients who have been diagnosed with a psychiatric condition and previous patients who have not been diagnosed with the psychiatric condition; processing the first collected electronic health records into a training dataset; and training a machine learning model with the training dataset to compute the specific patient's future risks for developing the psychiatric condition. In a more detailed embodiment, the processing step includes separating the collected electronic health records based on the age that the previous patients were diagnosed with the psychiatric disorder.

Alternatively, or in addition, the method further includes a step of collecting second electronic health records from previous patients who have not who have not been diagnosed with the psychiatric condition and processing the second electronic health records into a control dataset. In a further detailed embodiment, the steps of processing the first and/or second collected health records include extracting data relevant to the psychiatric condition from the records. In a further detailed embodiment, the extracted data includes structured constant data (e.g., fixed format data that remain constant over a patient's treatment, such as age, demographics, family medical history, etc.). Alternatively, or in addition, the extracted data includes structured longitudinal data (e.g., time-sequence fixed format data representing the patient's course of care, such as diagnosis codes, procedure codes, medication lists, types of providers and specialties seen, locations of doctor visits, amount of time spent in patient care, and/or biometrics such as height, weight, BMI, blood pressure and heart rate). Alternatively, or in addition, the extracted data includes environmental longitudinal data (e.g., time sequence data that represent the environment around the patient's place of residence over time, such as local childhood opportunity index, community deprivation index, American community survey, and metrics representing the amount of traffic exposure and greenspace in the area). Alternatively, or in addition, the extracted data includes school longitudinal data (e.g. absences, national test scores, suspensions). Alternatively, or in addition, the extracted data includes unstructured longitudinal data (e.g., features such as diagnoses, symptoms, treatments, and medications extracted from free-text clinical notes associated with hospital visits).

In an embodiment, the extracted data includes structured data and unstructured data. In an embodiment, the method includes a step of organizing the extracted data into time bins. In a more detailed embodiment, the step of organizing the extracted data into time bins includes: aligning each patient's extracted data from the date of the patient's first psychiatric condition (e.g. diagnosis, emergency room visit, hospitalization, suicide attempt) then counting backward by a series of fixed time intervals to create a time window of respective time bins of extracted data for each patient.

In an embodiment, the training step comprises training multiple machine learning models. In a further detailed embodiment, a machine learning model is trained for each age cohort (e.g., one model for patients with a first condition at year x, another machine learning model for patients with a first condition at year y, and so forth). In a further detailed embodiment, a machine learning model is trained by combining age cohorts.

In a further detailed aspect, the risk probability output includes the following steps: computing a probability of the psychiatric condition (e.g., diagnosis, emergency room visit, hospitalization, suicide attempt) over a series of future time windows and plotting or otherwise distributing the computed probabilities against future time windows. In a more detailed embodiment, the trajectory probability output includes adding all the probability percentages, dividing by the sum of all the sets, and multiplying by 100 to create a standardized score.

In another detailed aspect, the training step excludes the training dataset data from a predetermined blackout period. In a more detailed embodiment, the blackout period is a time period leading up to the training patient's psychiatric condition (e.g. diagnosis, emergency room visit, hospitalization, suicide attempt).

In a further detailed aspect, the processing step includes mitigating bias in the electronic health record data.

Another aspect of the current disclosure is directed to one or more machine learning models trained and prepared according to the above method(s).

Another aspect of the current disclosure is directed to a non-transitory memory device including computer instructions for directing one or more processors to perform any of the above method(s).

### BRIEF DESCRIPTION OF THE DRAWINGS

The drawings described below are for illustrative purposes only. The drawings are not intended to limit the scope of the present teachings in any way.

FIG. 1 illustrates a method that may be used to create software and/or used to train and prepare a machine learning model for use to compute the specific patient's future risks for developing the psychiatric condition (e.g., diagnosis, emergency room visit, hospitalization, suicide attempt).

FIG. 2 illustrates a process for obtaining a foundation dataset from an EHR database;

FIG. 3 illustrates how data may be binned across time;

FIG. 4 illustrates the results of using a machine-learning model to determine the effect of the number of health care visits on the mean classifier probability for a future diagnosis of an anxiety disorder

FIG. 5 illustrates a method that may be used to identify a specific patient's future risk of developing a psychiatric condition and then a clinician provider pro-active treatment options to the family;

FIG. 6 illustrates a graph showing one patient with five health care encounters (open circles) with corresponding risk trajectory scores that are based on both structured and unstructured data over time;

FIG. 7 illustrates a graph showing one patient with multiple health care encounters (open circles and filled circles) with corresponding risk trajectory scores, one line using only structured data, the other using both structured and unstructured data over time;

FIG. 8A is the general template where the X axis is months in the future using today as the starting point. The Y-axis is the probability that this specific patient will develop a specific psychiatric condition (in this example a diagnosis of an anxiety disorder). The cross-hatched horizontal bars represent domain experts' specific interpretation of the risk level for that psychiatric condition. In this example, the bottom hatching region represents a low risk of developing the condition (anxiety disorder), while the top hatching region represents a high risk of developing the condition (anxiety disorder). The middle hatching region represents an intermediate risk of developing the condition (anxiety disorder).

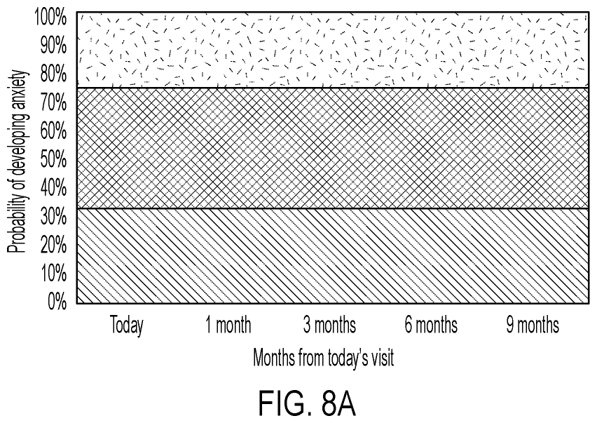

FIG. 8B shows a specific patient with a persistent low risk of developing an anxiety disorder.

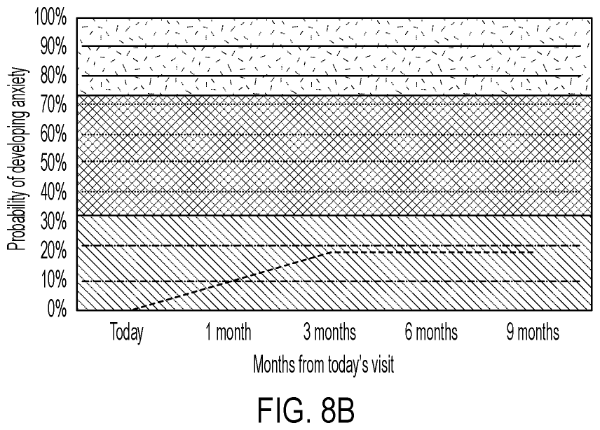

FIG. 8C shows a clear upward trajectory of the probability line indicating an increasing risk for the next 9 months ending with a high risk of developing an anxiety disorder.

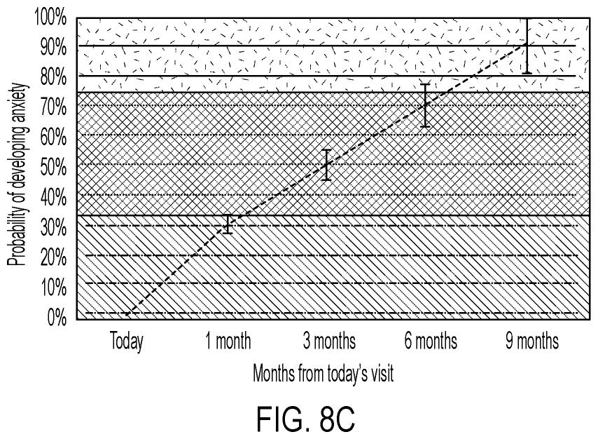

### DETAILED DESCRIPTION

Disclosed herein are techniques that may be used for identifying a specific patient's future risk of developing a psychiatric condition (e.g., diagnosis, emergency room visit, hospitalization, suicide attempt). However, these examples are intended to be illustrative only, and the disclosed techniques may be applied in other contexts, such as identifying and treating other psychiatric conditions. Accordingly, the examples provided here should not be treated as implying limitations on the protection provided by this document or by any other related document.

The methods may comprise, consist of, or consist essentially of the elements of the methods as described herein, as well as any additional or optional element described herein or otherwise useful in methods for training and preparing a machine learning model for use in identifying a specific patient's future risk of developing a psychiatric condition.

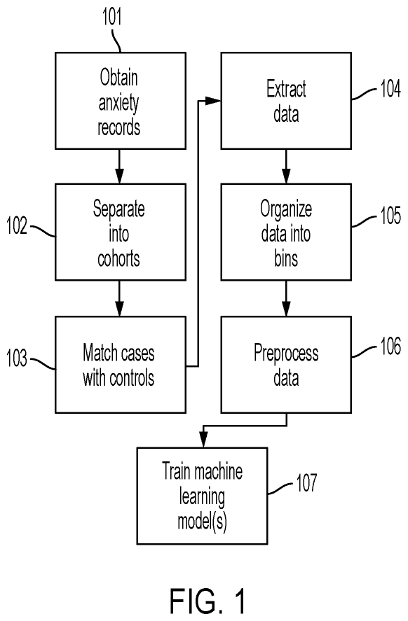

Turning first to FIG. 1. this figure illustrates a method that may be used to create software and/or used to train and prepare one or more machine learning models for use in the early identification and (earlier) treatment of a psychiatric condition, such as an anxiety disorder. As shown in FIG. 1, such a method may begin with obtaining 101, a set of electronic health records (EHRs) associated with the psychiatric condition. This may include using a computer to query an EHR repository for records of patients diagnosed with an anxiety disorder (e.g., as shown through the presence of ICD9 and/or ICD10 diagnosis codes) and then anonymizing those records to obtain an anonymized anxiety disorder dataset. This may also include applying other parameters in obtaining the dataset. For example, in a case where software was being created that would be used for identifying a specific patient's risk of developing an anxiety disorder, the dataset may be limited to only individuals who had not received an anxiety diagnosis. Hence, more specifically focus on what would happen prior to the diagnosis event.

Once the psychiatric disorder records have been obtained 101, they may be separated 102 into cohorts. This may be done, for example, by dividing the EHRs based on age at the time of diagnosis, such as by separating EHRs into 25 cohorts, one for individuals who were one year old at the time of first diagnosis, one for individuals who were two years old at the time of first diagnosis, etc., up until a cohort was created for individuals who were twenty-five years old at the time of first diagnosis. Following the separation of 102, the cases may be matched to 103 with corresponding controls. This may be done in several ways. For instance, EHRs for patients with ages and genders matching those from the case group but who had not received a diagnosis of the psychiatric condition may be retrieved from EHR system, anonymized, and then paired with the matching cases. Data that would be relevant to the psychiatric condition (e.g., anxiety diagnoses) could then be extracted 104 from the records. This may include extracting a variety of categories of data, such as structured constant data (e.g., fixed-format data that remain constant over a patient's treatment, such as age, demographics, family medical history, etc.), structured longitudinal data (e.g., time-sequence fixed-format data representing the patient's course of care, such as diagnosis codes, procedure codes, medication lists, types or providers and specialties seen, locations of doctor visits, amount of time spent in in-patient care, and/or biometrics such as height, weight, BMI, blood pressure and heart rate), environmental longitudinal data (e.g., time sequence data that represent the environment around the patient's place of residence over time, such as local childhood opportunity index, community deprivation index, American community survey, and metrics representing amount of traffic exposure and greenspace in the area), school longitudinal data (e.g. absences, national test scores, suspensions), and unstructured longitudinal data (e.g., features such as diagnoses, symptoms, treatments and medications extracted from free-text clinical notes associated with hospital visits, such as may be extracted using the SparkNLP software made available by John Snow Labs at https://nlp.johnsnowlabs.com/docs/en/jsl/install which features may be mapped to appropriate categories, such as ICD10 categories after extraction).

In the method of FIG. 1, once the data has been extracted 104 from the records, the extracted data could be organized 105 into bins, such as time bins. This may be done by aligning each case's extracted data from that case's first psychiatric condition (e.g., anxiety diagnosis), then counting backward by a series of fixed time intervals (i.e., bins) to create a time window of respective time bins for the patient's life from birth through diagnosis. Some or all of the extracted data (e.g., the longitudinal data only) could then be added to the bins based on which bins covered the date on which the various data items were added to (or changed in) the patient's record. The binned data may then be preprocessed 106 to prepare it for use in training 107 one or more machine learning model(s). The specific preprocessing applied may vary from one type of data to another. So, to illustrate what types of preprocessing may be applied, various examples of potential preprocessing steps are set forth below.

A dimensionality reduction is one type of preprocessing 106 that may be applied in a method such as illustrated in FIG. 1. This type of preprocessing may be performed in various ways. For example, data types represented in explicit ontologies (e.g., diagnosis codes, procedure codes, and/or medications) may have their dimensionality reduced by leveraging the structure of the relevant ontology. To illustrate, consider diagnostic information reflected in ICD10 codes. Because these codes are hierarchical, diagnostic codes may be rolled up from the most granular levels to lower levels of granularity when the most granular data is not needed. This may be done in a data-driven manner (e.g., by identifying the differences between cohorts and then rolling up items that present the lowest statistical differences) or in an expert-curated manner (e.g., subject matter experts specifying how blocks should be rolled up). Other approaches are also possible. For example, ICD10 information could be transformed into CCSR information (as described at https://hcup-us.ahrq.gov/toolssoftware/ccsr/ccs_refined.jsp) to reduce the dimensionality of the data.

Another type of preprocessing 106 which may be applied is bin encoding. For example, low dimensional representations of data (e.g., as a result of the data being mapped from high to low dimensionality as described above or because the data is inherently low dimensional, such as provider-type data) may be represented as frequency encoded columns per time-bin. Similarly, biometrics may be encoded as the average of a patient's percentiles per time bin (e.g., the mean of all weight measurement percentiles in a time bin). Further processing may also be applied to bin encoded data. For example, embedding techniques (e.g., principal component analysis to map binned data into lower dimensional space) may be applied to reduce the dimensionality of bin-level data to a size appropriate for the sparsity of the data, number of bins, and age cohort.

However it is performed, once the data is preprocessed 106, it could be used to train 107 one or more machine learning model(s). This may be done, for example, by individually training a model for each age cohort (e.g., one model for patients with a first diagnosis at one year, one model for individuals with a first diagnosis at two years, etc.) to generate a probability for when (if at all) individuals in that cohort are likely to be developed a psychiatric condition (e.g., diagnosed with an anxiety disorder). This could be done using a variety of machine learning model types, such as classic and deep learning methods, which would directly output a prediction, classifiers which would specify potential delays until diagnosis as various classes with various probabilities, decision trees such as XG Boost, or other types of machine learning models. These machine learning models could then be subjected to some level of validation (e.g., 10-fold cross-validation) and, once validated, could be incorporated into software stored in a non-transitory computer-readable medium and used to configure the processor of a computer to compute the specific patient's future risks for developing the psychiatric condition (e.g., diagnosis, emergency room visit, hospitalization, suicide attempt).

According to the current disclosure, the probability output is a probability likelihood and differs from inferential statistics, which use historical data to understand the past. Statistically, one could say that “tomorrow, there is a 20% chance that a patient with an anxiety disorder will come into the clinic.” Generally, statements like these use inferential statistics: they look at the frequency of an event that has occurred in the past and utilize an inference to apply it to the future. The clinician says I have seen this pattern 20% of the time. So, there is a 20% chance I will see a patient with an anxiety disorder tomorrow. Using this statistical inference approach, it can be interpreted that that patient may also receive multiple diagnoses of anxiety.

On the other hand, the invention's trajectory probability is a chance that some event may occur-vs.-prediction, which is about predicting what outcome might come based on the highest probability event. For example, in a specific embodiment, the current disclosure can be used to understand the likelihood of someone being diagnosed with a psychiatric condition (e.g., diagnosed with an anxiety disorder) within a predetermined future time frame (e.g., the next six months). Using a patient's electronic health record, a specific embodiment computes a probability of a discrete event (an anxiety disorder diagnosis) to determine the future likelihood that a patient will be diagnosed with an anxiety disorder.

Embodiments utilize the marginal probability because it is the probability of an event irrespective of the outcomes of other random variables, e.g., P(A). Subsequent (e.g., the next six months after the initial six months, the next six months after that, and so forth) marginal probabilities are computed, and the likelihoods are plotted to create a trajectory. To calculate an overall likelihood score, one embodiment calculates the average percentage by adding all percentages together as numbered values and dividing by the sum of all the data sets. Then, multiply that output by 100 to create a standardized score.

An understanding of disease trajectories gained from time series data is utilized to generate the trajectory risk probabilities. It may be important in certain embodiments, for example, to determine (a) how many stages of a condition exist per each condition, (b) how individuals may progress through stages of the condition differently, (c) the triggers for transitions between stages, and (d) how long individuals may stay in stages.

With such understanding, embodiments of the current disclosure can successfully identify the specific patient's future risks for developing the psychiatric condition (e.g., diagnosis, emergency room visit, hospitalization, suicide attempt) early based on changing patient characteristics, symptoms, and morbidities.

Diseases such as anxiety and depression progress throughout a patient's lifetime. This progression can be segmented into “stages” that manifest through clinical observations. A growing area in precision medicine is the forecasting of personalized disease trajectories using patterns in temporal correlations and associations between related diseases. The current disclosure aims to build disease progression models from electronic health records and other informative datasets, learn the model parameters at training time, and then issue personalized dynamic forecasts. In addition to providing accurate forecasts for the patient at hand, embodiments of the current disclosure will be able to make discoveries regarding disease progression mechanisms at the population, sub-group, and personalized levels. (M. van der Schaar and F. Imrie, Associating for the Advancement of Artificial Intelligence, Feb. 23, 2022).

The ability to identify a specific patient's future risks for developing a psychiatric condition such as pediatric anxiety can lead to better and more effective treatment, potentially eliminating a lifetime of misery or even death. Since diagnosis is the practice of inferring a recognizable pattern manifested by the patient's current signs and symptoms, identifying a specific patient's future risk of being diagnosed with a psychiatric condition requires correct understanding and characterization of the staging and likely trajectory of that disease. For example, stage one might begin as a toddler (12-18 months) may experience separation anxiety, whose relevant psychopathological symptoms include sleep disturbance, nocturnal panic attacks, and oppositional deviant behavior. The diagnostic classification may be separation anxiety disorder and/or panic attacks. The next stage of anxiety characteristics might be two/three-year-olds that fear thunder and lightning, fire, water, darkness, and nightmares. These anxiety characteristics can manifest as crying, clinging, withdrawal, freezing, seeking security and physical contact, avoidance of salient stimuli (e.g., turning the light on), pavor nocturnus, and enuresis. Some scholars have shown up to seven stages. Beesdo, K., Knappe, S., & Pine, D. S. (2009). Anxiety and Anxiety Disorders in Children and Adolescents: Developmental Issues and Implications for DSM-V. The Psychiatric Clinics of North America, 32(3), 483.
https://doi.org/10.1016/j.psc.2009.06.002.

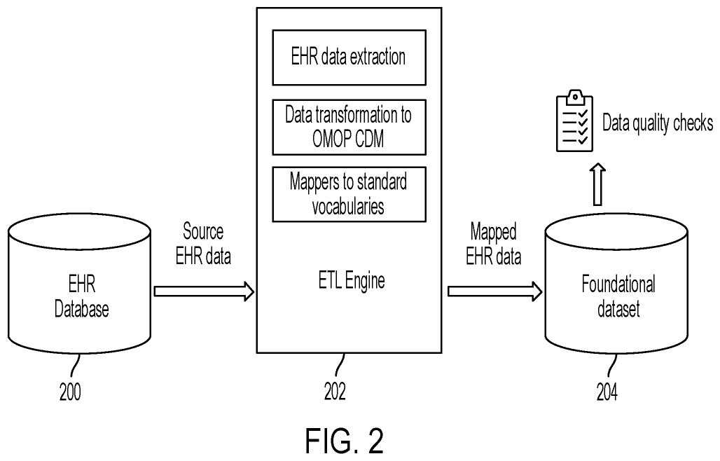

Of course, it should be understood that while FIG. 1 and the associated text described and illustrated a method that may be used to create software and/or train one or more ML models to compute a specific patient's future risks for developing a psychiatric condition (e.g. diagnosis, emergency room visit, hospitalization, suicide attempt) and how the steps of such a method may be performed, the description and illustration provided is intended to be illustrative only. Variations on the above description are both possible and will be immediately apparent to one of skill in the art in light of this disclosure. For example, in some cases, the step of obtaining 101 psychiatric condition (e.g., anxiety) records may include translating records from disparate systems into a common form, such as the common data model (CDM) from the observational medical outcomes partnership (OMOP). For example, as shown in FIG. 2, data from an EHR database 200 may be subjected to an extract, transform, load (ETL) process 202 in which vocabulary mapping is used to map terms from EHRs into the OMOP CDM, thereby providing a dataset (e.g., an anxiety dataset) 204 which could serve as a foundation for future analysis.

Referring back to FIG. 1, another example of a potential variation in the above discussion is in the implementation of matching cases with controls 103 and separating cases into cohorts 102. As set forth above, it is possible that cases may be separated into cohorts and then matched with controls. However, it is also possible that cases may initially be matched with controls (which matching may be 1:1 matching, 1:n matching, or other types of case: control matching). Then the case/control pairs could be separated into cohorts. In this type of approach, the cohorts may be created such that there are no repeated controls within any age cohort, or repetitions could be allowed, depending on factors such as the number of cases and matches. Accordingly, the above discussion of matching cases with controls 103 and separating cases into cohorts 102 should not be treated as implying limitations on how those steps may be performed, including on their order of performance.

Variations are also possible in how data would be extracted 104 for the cases and controls. For example, while the above disclosure provided examples of types of data that may be extracted, other types of data may also be used, such as allergies, which may be identified by training a natural language processing model to learn and identify categories of allergies (e.g., food, medications, and environment) from text notes included in patient EHRs. Similarly, comorbidities may be extracted and represented using codes such as ICD9 and ICD10 codes. Data extraction 104 may also include various types of transformations. For example, procedures may be represented as procedure codes, which may be mapped to categories in the clinical classifications software (CCS) for Services and Procedures Tool Provided by the Agency for Healthcare Research and Quality.

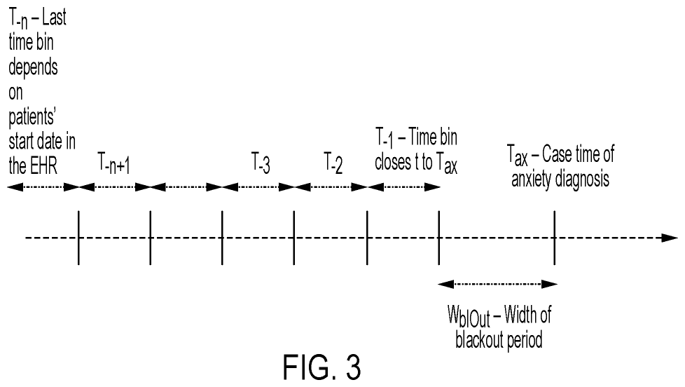

The organization of data into bins 105 may also vary between implementations of the disclosed technology. For example, bin sizes may vary and represent durations such as 5, 10, 15, 20, years or 15 days, 30 days, 60 days, 183 days (~6 months), 365 days, or other durations. In some cases, there may be a blackout period for data, which would be excluded from training a machine learning model. This may be the bin closest to a case's psychiatric condition (e.g., anxiety diagnosis) or may be a different period. This is illustrated in FIG. 3, which shows how data for a case may be binned, with each time bins extending from T₋ₙ, T₋ₙ₊₁ to T₋₁, and the blackout period shows as a period of width W_{blOut} ending at Tₐₓ.

Variation in data is also possible, not just in how steps such as shown in FIG. 1 could be implemented, but also in whether more (or fewer) steps are used in creating software to compute the specific patient's future risks for developing the psychiatric condition (e.g. diagnosis, emergency room visit, hospitalization, suicide attempt). For example, in some cases, in addition to extracting data 104 from EHRs, data may be retrieved from external sources that provide a more complete picture. For example, for training machine learning models using environmental data, if an EHR did not include relevant environmental data (e.g., childhood opportunity index data), then this information may be obtained by retrieving it from an external database using information from the EHR (e.g., patient zip code) as a query parameter. As another example, in some cases, there may be an additional step (e.g., following obtaining anxiety records 101) of assessing data quality and potentially modifying the dataset based on the quality assessment.

One quality measure is bias. Predictive bias is a situation in which AI is found to give systematically different predictions for subgroups of the population that are, in reality, identical. For example, focusing on the analysis of the selection bias in the free text clinical notes of anxiety patients, it was found that Black/Afro-American and Other race groups are under-represented in terms of the number of patients (around 16% for each group, with the rest of the patients being White). The ratio of women and men is more balanced (40% vs. 60%). Differences in topics discussed in the notes of the Black/Afro-American and White race groups were also observed. These topics concern diagnostic conditions the patients suffer from (e.g., conditions requiring imaging vs. ear and eye conditions). Those differences in the original data, however, do not result in substantial bias across age and demographic groups in the state-of-the-art AI classifiers (Transformer-based models) trained to predict anxiety diagnosis (as measured with a balanced error rate [1]). Co-pending U.S. Application No. 63/583,707, filed Sep. 19, 2023 and incorporated herein by reference, provides systems and methods for identifying and mitigating bias in clinical artificial intelligence models. It would be within the scope of the present disclosure to utilize any of the systems and methods disclosed therein for mitigating bias in the EHR data in the datasets of the current disclosure.

Accordingly, the discussion of the steps that could be included in a method for creating software to compute the specific patient's future risks for developing the psychiatric condition (e.g. diagnosis, emergency room visit, hospitalization, suicide attempt), like the discussion of how such steps may be performed, should be understood as being illustrative only, and should not be treated as limiting.

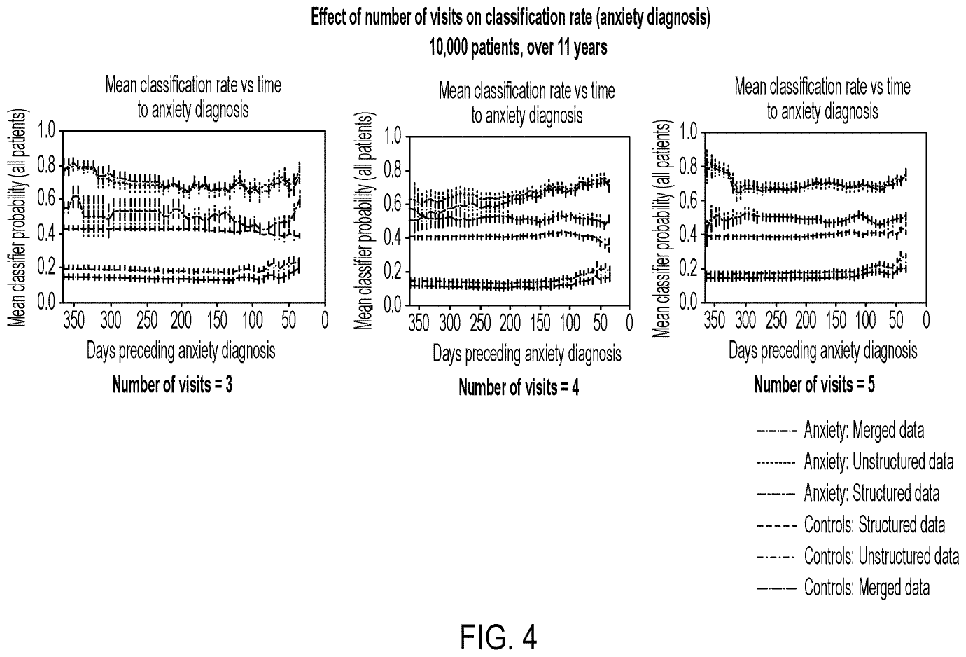

To illustrate how software using the method shown in FIG. 1 may be applied to determine the effect of the number of health care visits on the mean classifier probability for a future diagnosis of an anxiety disorder, consider the results shown in FIG. 4. By applying a system implemented using the technology described herein, trajectories of future probabilities of an anxiety disorder diagnosis were generated based on a dataset of 10,000 patients with data gathered over 11 years and using structured and unstructured data such as described herein. The trajectories demonstrated that with as few as 5 health care visits of any type, it was possible to accurately separate individuals destined to be diagnosed with an anxiety disorder 50 days (on average) before the actual anxiety disorder diagnosis from those in the control cohort.

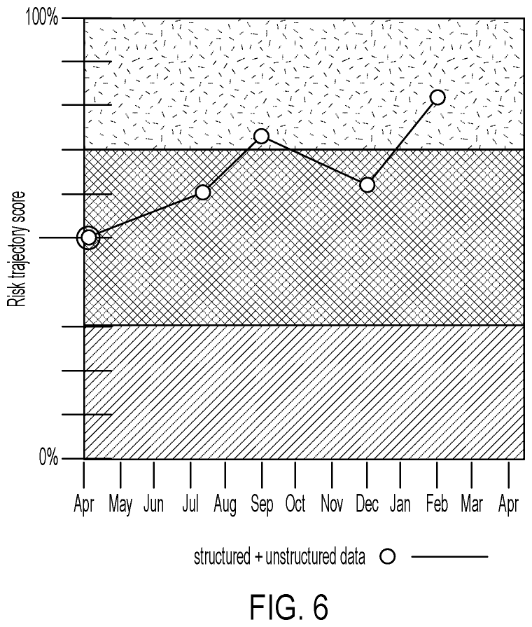

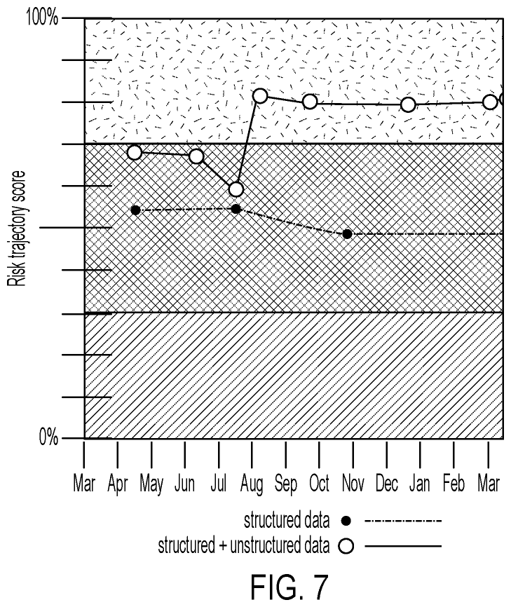

FIG. 6 illustrates a graph showing one patient with five health care encounters (open circles) with corresponding risk trajectory scores that are based on both structured and unstructured data over time. It is an example of output combining structured and unstructured data that increases from the intermediate to high-risk regions over time. FIG. 7 illustrates a graph showing one patient with multiple health care encounters (open circles and filled circles) with corresponding risk trajectory scores, one line using only structured data, the other using both structured and unstructured data over time. This example shows that adding unstructured data provides significant additional information that could change a provider's course of action.

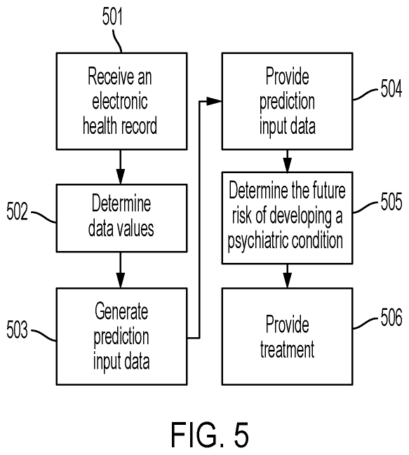

Software created that includes a method such as that shown in FIG. 1 may also be applied to change the treatment approach based on identifying a specific patient's future risk of developing a psychiatric condition (e.g. diagnosis, emergency room visit, hospitalization, suicide attempt). A method of treatment that may be performed using such software is illustrated in FIG. 5. Initially, in that method, an electronic health record would be received 501 for an individual. This may be done, for example, by a treating physician retrieving a medical record for a patient under his or her care from a medical record system. After the electronic health record had been retrieved 501, it could be used to determine 502, a plurality of data values. This data value determination 502 may be performed, for example, by using natural language processing software to extract unstructured longitudinal data or otherwise using techniques such as those described previously in the context of training to retrieve information from the electronic health record. Once the data values had been determined 502, the method of FIG. 5 continues with the generation 503 of prediction input data. This may include processing the data from the electronic health record like that discussed previously in the context of training (e.g., normalizing and organizing the data into time-limited bins) to put it in the same form as the data used to train the machine learning model. Once the data was in the form that the machine learning model had been trained to use as input, it could be provided 504 to the trained machine learning model, and, based on the output of that model, the individual could be identified 505 based on the trajectory output as having a high, intermediate, or low future risk of developing a psychiatric condition (e.g. diagnosis, emergency room visit, hospitalization, suicide attempt). Finally, if the patient was identified as having a high risk of developing a psychiatric condition needing treatment, they could be provided 506 with treatment, such as by being treated using cognitive behavioral therapy, another similar evidence based behavioral therapy and/or using pharmacological treatment such as (a) a selective serotonin reuptake inhibitor; or (b) serotonin-norepinephrine reuptake inhibitor; or (c) benzodiazepines; or (d) beta blockers; or (e) tricyclic antidepressants; or (f) monoamine oxidase inhibitors; or (g) atypical antidepressants; or (h) lithium; or (i) antiepileptics; or (j) first, second, or third generations antipsychotics; or (k) stimulants; or (l) non-stimulant (atomoxetine, clonidine, guanfacine, and modafinil); or (m) non-benzodiazepine anxiolytics.

The current disclosure provides machine learning models for identifying a specific patient's future risk of developing a psychiatric condition (e.g. diagnosis, emergency room visit, hospitalization, suicide attempt) and systems and methods for developing and/or training such machine learning models. While anxiety is the primary exemplary psychiatric disorder discussed herein, it will be apparent that it is within the scope of the current disclosure to use the systems and methods herein for use with other psychiatric disorders such as depression, suicide risk, and risk of violent behavior.

The computing engines, modules, machine learning modules, machine learning engines, deep learning modules/engines, training systems, architectures and other disclosed functions are embodied as computer instructions that may be installed for running on one or more computer devices and/or computer servers. In some instances, a local user can connect directly to the system; in other instances, a remote user can connect to the system via a network.

Example networks can include one or more types of communication networks. For example communication networks can include (without limitation), the Internet, a local area network (LAN), a wide area network (WAN), various types of telephone networks, and other suitable mobile or cellular network technologies, or any combination thereof. Communication within the network can be realized through any suitable connection (including wired or wireless) and communication technology or standard (wireless fidelity (WiFi®), 4G, 5G, long-term evolution (LTE™)), and the like as the standards develop.

The computer device(s) and/or computer server(s) can be configured with one or more computer processors and a computer memory (including transitory computer memory and/or non-transitory computer memory), configured to perform various data processing operations. The computer device(s) and/or computer server(s) also include a network communication interface to connect to the network(s) and other suitable electronic components.

Example local and/or remote user devices can include a personal computer, portable computer, smartphone, tablet, notepad, dedicated server computer devices, any type of communication device, and/or other suitable compute devices.

The computer device(s) and/or computer server(s) can include one or more computer processors and computer memories (including transitory computer memory and/or non-transitory computer memory), which are configured to perform various data processing and communication operations associated with diagnosing psychiatric disorders as disclosed herein based upon information obtained/provided (such as the EHR data discussed above) over the network, from a user and/or from a storage device. In some implementations, storage device can be physically integrated to the computer device(s) and/or computer server(s); in other implementations, storage device can be a repository such as a Network-Attached Storage (NAS) device, an array of hard-disks, a storage server or other suitable repository separate from the computer device(s) and/or computer server(s).

In some instances, storage device can include the machine-learning models/engines and other software engines or modules as described herein. Storage device can also include sets of computer executable instructions to perform some or all the operations described herein.

### Definitions

Unless otherwise noted, terms will be understood according to conventional usage by those of ordinary skill in the relevant art. The present document, including definitions, will control in case of conflict. Preferred methods and materials are described below, although methods and materials similar or equivalent to those described herein may be used in practice or testing of the present invention. All publications, patent applications, patents, and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context dictates otherwise. Thus, for example, reference to “a method” includes one or more such methods, and reference to “a machine learning model” includes reference to one or more machine learning models and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within one or more than one standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims unless otherwise stated, the term “about,” meaning within an acceptable error range for the particular value, should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially, or simultaneously.

The terms “individual,” “host,” “subject,” and “patient” are used interchangeably to refer to the human that is the object of treatment, observation, and/or experiment. Generally, the term refers to a human patient. In some embodiments, the terms may refer to children.

As used herein, “based on” should be understood to mean that a thing is determined, at least in part, by that which it is indicated as being “based on.” It should be understood that a statement that something is “based on” something else does not necessarily require one thing to be fully determined by the other. If one thing is required to be fully determined by another, this may be indicated by stating that it is “based EXCLUSIVELY on” that which it is determined by.

“Longitudinal data” refers to data taken, collected, distributed or provided over a period of time. This is opposed to cross-sectional data, which is data with respect to a point in time. In other words, longitudinal data is the history of data as opposed to data in a point in time.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention and thus may be considered to constitute examples of modes for its practice. However, those of skill in the art should appreciate that many changes may be made in the specific embodiments that are disclosed and still obtain a similar result without departing from the spirit and scope of the invention.

### Example 1

A method for identifying a specific patient's future risk of developing a psychiatric condition (e.g. diagnosis, emergency room visit, hospitalization, suicide attempt), comprising: (a) receiving an electronic health record for an individual; (b) based on the electronic health record, determining a plurality of data values, wherein the plurality of data values comprise a set of unstructured longitudinal data values; (c) generating trajectory input data based on performing a set of processing activities on the plurality of data values, wherein the set of processing activities comprises: (i) normalizing the set of unstructured longitudinal data values, and (ii) organizing the set of unstructured longitudinal data values into a set of time-limited bins; (d) providing the trajectory input data to a trained machine learning model; (e) based on an output of the trained machine learning model, identifying the individual as being low, intermediate or high risk for developing a psychiatric condition; and (f) based on identifying the individual as high risk for developing a psychiatric condition, providing treatment to the individual.

### Example 2

The method of example 1, wherein providing the treatment to the individual comprises treating the individual with cognitive behavioral therapy or another similar evidence based behavioral therapy.

### Example 3

The method of example 1, wherein providing the treatment to the individual comprises treating the individual with a medication selected from appropriate medications for the psychiatric condition which could include (a) a selective serotonin reuptake inhibitor; and (b) serotonin-norepinephrine reuptake inhibitor; and (c) benzodiazepines; and (d) beta blockers; and (e) tricyclic antidepressants; and (f) monoamine oxidase inhibitors; and (g) atypical antidepressants; and (h) lithium; and (i) antiepileptics; and (j) first, second, or third generations antipsychotics; and (k) stimulants; and (1) non-stimulant (atomoxetine, clonidine, guanfacine, and modafinil); and (m) non-benzodiazepine anxiolytics.

### Example 4

The method of any preceding example, wherein the plurality of data values comprises (a) a set of structured constant data values; (b) a set of unstructured constant data values; (c) a set of structure longitudinal data values; (d) a set of unstructured longitudinal data values; (e) a set of environmental longitudinal data values; and (f) a set of school longitudinal data values.

### Example 5

The method of any preceding example, wherein the individual is a pediatric patient.

### Example 6

The method of any preceding example, wherein the individual is between five and ten years old. Alternatively, the method of any preceding example, wherein the individual is between two and five years old.

### Example 7

The method of any preceding example, wherein the individual is between ten and fifteen years old. Alternatively, the method of any preceding example, wherein the individual is between fifteen and twenty-five years old.

### Example 8

The method of any preceding example, wherein the individual is an adolescent patient or the individual is a young adult patient less than or equal to 25 years of age.

### Example 9

The method of any preceding example, wherein the psychiatric condition comprises a likelihood of developing within a predetermined number of days (e.g. 30, 60, 09, 120, 180, 270, 365 days) of the machine learning model output for the specific patient's future risk of developing the psychiatric condition

### Example 10

The method of example 9, wherein the anxiety disorder has an ICD-9 diagnosis code of ‘300.0’, ‘300.01’, ‘300.02’, ‘300.21’, ‘300.22’, ‘300.23’, or ‘309.21’, or ICD-10 diagnosis codes of ‘F40.01’, ‘F40.02’, ‘F40.10’, ‘F40.11’, ‘F41.0’, ‘F41.1’. ‘F41.9’, or ‘F93.0’; the depression disorder has an ICD-9 diagnosis code of ‘296.2’, ‘296.20’, ‘296.21’, ‘296.22’, ‘296.23’, ‘296.24’, ‘296.25’, ‘296.26’, ‘296.3’, ‘296.30’, ‘296.31’, ‘296.32’, ‘296.33’, ‘296.34’, ‘296.35’, ‘296.36’, ‘296.82’, ‘296.99’, ‘300.4’, ‘301.12’, ‘309.0’, ‘309.1’, or ICD-10 diagnosis codes of ‘F32.0’, ‘F32.1’, ‘F32.2’, ‘F32.3’, ‘F32.4’, ‘F32.5’, ‘F32.8’, ‘F32.81’, ‘F32.89’, ‘F32.9’, ‘F32.A’, ‘F33.0’, ‘F33.1’, ‘F33.2’, ‘F33.3’, ‘F33.40’, ‘F33.41’, ‘F33.42’, ‘F33.8’, ‘F33.9’, ‘F34.1’, ‘F43.21’; the suicidal ideation ICD-9 diagnosis code of ‘V62.84’ or ICD-10 diagnosis code of ‘R45.851’, ‘T14.91’, ‘T14.91XA’, ‘T14.91XD’, or ‘T14.91XS’.

### Example 11

The method of any preceding example, wherein the steps of clauses (a)-(f) of example 1 are performed before the individual being diagnosed with a psychiatric condition including but not limited to an anxiety, depressive, or suicidal disorder.

### Example 12

The method of any preceding example, wherein (a) the trained machine learning model is configured to provide classifications of low risk of developing a psychiatric condition, intermediate risk of developing a psychiatric condition, and high risk of developing a psychiatric condition; and; and (b) the method comprises identifying the individual as having a high risk of a future psychiatric condition based on the trained machine learning model classifying the individual as high risk of developing the clinical disorder.

All percentages and ratios are calculated by weight unless otherwise indicated.

Unless otherwise indicated, all percentages and ratios are calculated based on the total composition.

It should be understood that every maximum numerical limitation given throughout this specification includes every lower numerical limitation, as if it were expressly written herein. Every minimum numerical limitation given throughout this specification will include every higher numerical limitation as if such higher numerical limitations were expressly written herein. Every numerical range given throughout this specification will include every narrower numerical range that falls within such broader numerical range as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross-referenced or related patent or application, is hereby incorporated herein by reference in its entirety unless expressly excluded or otherwise limited. All accessioned information (e.g., as identified by PUBMED, PubChem, NCBI, UniProt, or EBI accession numbers) and publications in their entireties are incorporated into this disclosure by reference to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is, therefore, intended to cover in the appended claims all such changes and modifications that are within the scope of this invention.

1. A method for training and preparing a machine learning model for use in identifying a specific patient's future risk of developing a psychiatric condition comprising:
collecting first electronic health records from previous patients who have been diagnosed with the psychiatric condition and a second electronic health records from previous patients who have not been diagnosed with the psychiatric condition;
processing the first and second collected electronic health records into a training dataset; and
training a machine learning model with the training dataset to compute the specific patient's future risks for developing the psychiatric condition.

2. The method of claim 1, wherein the processing step includes separating the first and second collected electronic health records based on the age that the previous patients were diagnosed with the psychiatric condition.

3. The method of claim 2, further comprising processing the second electronic health records into a control dataset.

4. The method of claim 3, wherein the steps of processing the first and/or second collected health records include extracting data relevant to the psychiatric disorder from the records.

5. The method of claim 4, wherein the extracted data includes structured constant data.

6. The method of claim 4, wherein the extracted data includes structured longitudinal data.

7. The method of claim 4, wherein the extracted data includes non-electronic health records data such as environmental and school longitudinal data.

8. The method of claim 4, wherein the extracted data includes unstructured longitudinal data.

9. The method of claim 4, wherein the extracted data includes structured data and unstructured data.

10. The method of claim 4, further comprises organizing the extracted data into time bins.

11. The method of claim 10, wherein the step of organizing the extracted data into time bins includes:
aligning each patient's extracted data from the date of the patient's first psychiatric condition; and then
counting backward by a series of fixed time intervals to create a time window of respective time bins of extracted data for each patient.

12. The method of claim 1, wherein the training step comprises training multiple machine learning models.

13. The method of claim 12, wherein a machine learning model is trained for each of a plurality of age cohorts.

14. The method of claim 13, wherein the machine learning models are trained by combining one or more of the plurality of age cohorts.

15. The method of claim 1 wherein the output includes the following steps:
computing a probability of the likelihood of a psychiatric condition being identified over a series of future time windows and
plotting or distributing the computed probabilities over the future time windows.

16. The method of claim 15 wherein the trajectory probability output includes adding all the probability percentages, dividing by the sum of all the sets, and multiplying by 100 to create a standardized score.

17. The method of claim 1, wherein the training step excludes the training dataset data from a predetermined blackout period.

18. The method of claim 17, wherein the blackout period is a time period leading up to the previous patient's diagnosis of the psychiatric disorder.

19. The method of claim 1 wherein the processing step includes mitigating bias in the electronic health record data.

20. The method of claim 1 wherein the output is a specific patient's probability of developing a psychiatric condition within a specific future time frame.

21. The method of claim 20, wherein the machine learning engine is trained and configured to compute a probability of a psychiatric condition to determine the future likelihood that a patient will be diagnosed with or experience the psychiatric condition.

22. A machine learning model trained and prepared according at least the following steps including:
collecting first electronic health records from previous patients who have been diagnosed with the psychiatric condition and a second electronic health records from previous patients who have not been diagnosed with the psychiatric condition;
processing the first and second collected electronic health records into a training dataset; and
training a machine learning model with the training dataset to compute the specific patient's future risks for developing the psychiatric condition.

23. A non-transitory memory device, including computer instructions for directing one or more processors to perform steps of:
collecting first electronic health records from previous patients who have been diagnosed with the psychiatric condition and a second electronic health records from previous patients who have not been diagnosed with the psychiatric condition;
processing the first and second collected electronic health records into a training dataset; and
training a machine learning model with the training dataset to compute the specific patient's future risks for developing the psychiatric condition.

24. A method for treating a future psychiatric condition, comprising:
a) receiving an electronic health record and corresponding non-electronic health records data, such as environmental and/or school performance data, for an individual;
b) based on the electronic health record data and non-electronic health records data, determining a plurality of data values, wherein the plurality of data values comprise a set of combined structured and unstructured longitudinal data values;
c) generating prediction input data based on performing a set of processing activities on the plurality of data values, wherein the set of processing activities comprises:
i) normalizing the set of unstructured longitudinal data values; and
ii) organizing the set of unstructured longitudinal data values into a set of time limited bins;
iii) creating the input data by combining the structured and unstructured data from both the electronic health record data and corresponding non-electronic health records data
d) providing the input data to train a machine learning model;
e) based on an output of the trained machine learning model, identifying the future probability that the individual will develop a psychiatric condition; and
f) based on identifying the individual as having a high risk for developing a future psychiatric condition, providing a treatment to the individual.

25. The method of claim 24, wherein providing the treatment to the individual comprises treating the individual with cognitive behavioral therapy or another similar evidence based behavioral therapy.

26. The method of claim 24, wherein providing the treatment to the individual comprises treating the individual with a medication selected from: (a) a selective serotonin reuptake inhibitor; (b) serotonin-norepinephrine reuptake inhibitor; (c) benzodiazepines; (d) beta blockers; (e) tricyclic antidepressants; (f) monoamine oxidase inhibitors; (g) atypical antidepressants; (h) lithium; (i) antiepileptics; (j) first, second, or third generations antipsychotics; (k) stimulants; (l) non-stimulant, such as atomoxetine, clonidine, guanfacine, and/or modafinil; and (m) non-benzodiazepine anxiolytics.

27. The method of claim 24, wherein the plurality of data values comprises: (a) a set of structured constant data values; (b) a set of unstructured constant data values; (c) a set of structure longitudinal data values; (d) a set of unstructured longitudinal data values; (e) a set of environmental longitudinal data values; and (f) a set of school longitudinal data values.

28. The method of claim 24, wherein the individual is a pediatric patient or the individual is an adolescent patient or the individual is a young adult patient less than or equal to 25 years of age.

29. The method of claim 24, wherein the psychiatric condition comprises a likelihood of developing within a predetermined number of days of the machine learning model output for the specific patient's future risk of developing the psychiatric condition.

30. The method of claim 24, wherein:
a) the trained machine learning model is configured to provide classifications of low risk of developing a psychiatric condition, intermediate risk of developing a psychiatric condition, and high risk of developing a psychiatric condition; and
b) the method comprises identifying the individual as having a high risk of a future psychiatric condition based on the trained machine learning model classifying the individual as high risk of developing the clinical disorder.

31. A non-transitory memory device, including computer instructions for directing one or more processors to perform steps including:
a) receiving an electronic health record and corresponding non-electronic health records data, such as environmental and/or school performance data, for an individual;
b) based on the electronic health record data and non-electronic health records data. determining a plurality of data values, wherein the plurality of data values comprise a set of combined structured and unstructured longitudinal data values;
c) generating prediction input data based on performing a set of processing activities on the plurality of data values, wherein the set of processing activities comprises:
i) normalizing the set of unstructured longitudinal data values; and
ii) organizing the set of unstructured longitudinal data values into a set of time limited bins;
iii) creating the input data by combining the structured and unstructured data from both the electronic health record data and corresponding non-electronic health records data
d) providing the input data to train a machine learning model;
e) based on an output of the trained machine learning model, identifying the future probability that the individual will develop a psychiatric condition; and
f) based on identifying the individual as having a high risk for developing a future psychiatric condition, providing a treatment to the individual.
